(19) United States
(12) Patent Application Publication
(10) Pub. No.: US 2026/0248699 A1
(43) Pub. Date: Aug. 27, 2026

(54) CARE AND/OR MAKE-UP COLORED COMPOSITION

(71) Applicant: L V M H RECHERCHE, SAINT JEAN DE BRAYE (FR)

(72) Inventors: Aurore VERLOO, SAINT JEAN DE BRAYE (FR); Audrey DESTREM, SAINT JEAN DE BRAYE (FR); Sylvie BOURDILLAULT, SAINT JEAN DE BRAYE (FR)

(73) Assignee: L V M H RECHERCHE, SAINT JEAN DE BRAYE (FR)

(21) Appl. No.: 18/854,423

(22) PCT Filed: Apr. 5, 2023
(86) PCT No.: PCT/FR2023/050488

§ 371 Date: Oct. 4, 2024

(30) Foreign Application Priority Data
Apr. 5, 2022 (FR) FR2203101

(51) Int. Cl. A61K 8/27; A61K 8/02; A61Q 1/04; A61Q 1/06; A61Q 1/08; A61Q 1/10

(52) U.S. Cl. CPC A61K 8/27; A61K 8/022; A61K 8/0229; A61Q 1/04; A61Q 1/06; A61Q 1/08; A61Q 1/10; A61K 2800/43

(57) ABSTRACT

The present invention relates to a preferably anhydrous care and/or make-up cosmetic composition for keratinous materials, in particular for the skin, eyelids, eyelashes or lips, the composition comprising, in a physiologically compatible medium: (i) zinc oxide; and (ii) one or more light-sensitive colourants chosen from the group made up of colourants and organic pigments, composite pigments comprising at least one of the colourants or organic pigments, pearlescent pigments comprising at least one of the colourants or organic pigments, and mixtures thereof.

### FIELD OF THE INVENTION

The present invention relates to the cosmetic field and in particular to colored care and/or make-up cosmetic compositions for keratinous materials, in particular for the skin, eyelids, eyelashes or lips.

### STATE OF THE ART

Colored anhydrous skin or lip care and/or make-up products are sought after by consumers for their coverage and staying power properties, and with added color for a skin and/or lip make-up effect.

Coverage is generally provided by white pigments and/or fillers, and titanium dioxide is used in particular for its strong opacifying and coverage power. Color is provided by organic and/or inorganic coloring materials, some of which are known to be light sensitive. This sensitivity can lead to instability of the surface color or shade when the composition is exposed to light. The color or shade on the surface of the composition exposed to light loses intensity and/or saturation; the surface of the composition is also said to be discolored in the light. It is therefore sought to preserve and protect light-sensitive coloring materials to avoid these variations in shade or color.

Unexpectedly, the Applicant has demonstrated that the use of zinc oxide particles, in particular in an anhydrous composition comprising light-sensitive coloring materials, allowed to protect said coloring materials and thus promoted the stability of the color and shade of the composition. Zinc oxide particles are therefore particularly advantageous, as a replacement for titanium dioxide particles, in the formulation of care and/or make-up compositions to provide coverage and also protect light-sensitive coloring materials. The use of these zinc oxide particles therefore allows to obtain stable shades, even with light-sensitive pigments, which was not possible with titanium dioxide.

### DISCLOSURE OF THE INVENTION

A first aspect of the invention is therefore a preferably anhydrous care and/or make-up cosmetic composition for keratinous materials, in particular for the skin, eyelids, eyelashes or lips, the composition comprising, in a physiologically acceptable medium:
(i) Zinc oxide particles, and
(ii) One or more light-sensitive coloring materials, chosen from the group consisting of colorants and organic pigments, composite pigments comprising at least one of said colorants or organic pigments, pearlescent pigments comprising at least one of said colorants or organic pigments, and mixtures thereof.

According to a particular and preferred embodiment, the composition of the invention is an anhydrous composition.

The present invention also relates to a cosmetic method for caring for and/or making up keratinous materials, in particular the skin, the eyelids, the eyelashes or the lips, preferably the lips, comprising the application to said keratinous materials of a composition according to the invention.

### DETAILED DESCRIPTION OF THE INVENTION

The present invention therefore relates to a preferably anhydrous care and/or make-up cosmetic composition for keratinous materials, in particular for the skin, eyelids, eyelashes or lips, the composition comprising, in a physiologically acceptable medium:
(i) Zinc oxide particles, and
(ii) One or more light-sensitive coloring materials, chosen from the group consisting of colorants and organic pigments, composite pigments comprising at least one of said colorants or organic pigments, pearlescent pigments comprising at least one of said colorants or organic pigments, and mixtures thereof.

“Anhydrous composition” means in particular that water is preferably not deliberately added to the composition of the invention but may be present in trace amounts in the various compounds used in the composition. In particular, the composition according to the invention comprises less than 4% by weight of water, preferably less than 3%, preferably less than 2%, more preferably less than 1%, even more preferably less than 0.5% by weight of water, relative to the total weight of said composition, or even is completely free of water. According to a particular embodiment, the composition is free of water.

According to one embodiment, the composition is fluid or liquid, that is to say it flows under its own weight at room temperature and atmospheric pressure.

According to another embodiment, the composition is in the form of a powder, in particular a loose powder.

According to another embodiment, the composition is a solid composition. Preferably the solid composition comprises one or more waxes.

“Solid composition”, as opposed to liquid or fluid composition or free powder, according to the invention means a composition which does not flow under its own weight at room temperature and atmospheric pressure.

The composition may in particular be in the form of a compact powder or a stick or a composition packaged in a cup.

“Compact powder” means a solid composition based on powders, different from a loose powder. The compact powder is generally prepared by mixing all the components of the particulate phase then adding a liquid phase to this mixture with stirring. The mixture is then poured into a cup, bucket or mold and compacted. Compaction is typically carried out at a pressure of 5 to 25 MPa, and results in a reduction in the volume of the compacted composition.

### Powdery Phase

The powdery phase of the composition comprises fillers and pigments.

The composition of the invention comprises in particular zinc oxide particles.

### Zinc Oxide Particles

Zinc oxide particles appear as a white powder and are insoluble in water.

Zinc oxide particles can be in a heterogeneous form, generally hexagonal and cubic.

The zinc oxide particles used according to the invention are pigmentary zinc oxide particles, as opposed to the zinc oxide nanoparticles used as a mineral sunscreen.

The average size of the zinc oxide particles used according to the invention will generally range from 0.1 to 50 μm (micrometers), in particular from 0.1 to 10 μm, or even from 0.1 to 1 μm.

The particle size can be measured by laser diffraction distribution method with a Malvern Mastersizer type apparatus from Malvern, in particular by measuring the value D[50], which represents the maximum size represented by 50% by volume of the particles.

Mention can in particular be made of the commercial references RONACARE ZINC OXIDE from the company Merck, or the reference A120-ZnO-11S3 from the company Kobo, or the reference ZINC OXIDE COSMETICS from the company KeyserEtMackay/Grillo.

According to a particular and preferred embodiment, the zinc oxide particles contain more than 80% by weight of ZnO, in particular more than 95%, 96%, 97%, 98%, or 99% by weight of ZnO, or even 100% ZnO.

The zinc oxide particles used according to the invention may or may not be treated with a lipophilic treatment agent for better dispersion in the fatty phase.

According to a particular embodiment of the invention, the zinc oxide particles are not treated and they have the INCI name: Zinc oxide.

According to another particular embodiment, the zinc oxide particles are treated with a lipophilic treatment agent.

The zinc oxide particles can also be mixed with a wetting agent to facilitate their dispersion in the composition and thus improve the homogeneity of the composition.

According to a particular embodiment, the content of zinc oxide particles in the composition of the invention will generally range from 0.1% to 50% by weight of zinc oxide, in particular from 1% to 30% by weight of zinc oxide, preferably from 2% to 20% by weight of zinc oxide relative to the total weight of the composition.

For a compacted powder composition (for example eye shadow, foundation powder), the total content of zinc oxide particles will generally range from 1% to 50%, preferably from 5% to 40% by weight (of zinc oxide) relative to the total weight of the composition.

For an anhydrous solid composition for the skin and different from a powder (for example complexion corrector), the total content of zinc oxide particles will generally range from 0.5% to 30%, preferably from 1% to 20% by weight (of zinc oxide) relative to the total weight of the composition.

For an anhydrous solid composition for the lips (for example stick for the lips), the total content of zinc oxide particles will generally range from 0.1% to 30%, preferably from 0.5% to 20% (of zinc oxide) by weight relative to the total weight of the composition.

According to a particular and preferred embodiment of the invention, the composition comprises less than 3% by weight of titanium dioxide relative to the total weight of the composition, in particular less than 1%, or is even free of titanium dioxide.

According to an even more preferred embodiment, the composition of the invention is free of titanium dioxide.

The composition further comprises one or more light-sensitive coloring materials.

### Light-Sensitive Coloring Materials

The term “coloring material” within the meaning of the present invention means a compound capable of producing a colored optical effect when formulated in sufficient amount in an appropriate cosmetic medium. The coloring materials are conventionally chosen from pigments (insoluble in oils and in the aqueous phase) and colorants (soluble in oils or in the aqueous phase).

The coloring materials may consist of material of mineral, synthetic or natural origin.

According to a particular embodiment, the coloring materials are of mineral or synthetic origin.

Said coloring materials may or may not be treated with a lipophilic treatment agent.

A colorant which is light sensitive and usable in the compositions of the invention can be chosen from the group consisting of colorants and organic pigments and/or their salts, the composite pigments comprising at least one of said colorants or organic pigments, pearlescent pigments comprising at least one of said colorants or organic pigments, and mixtures thereof.

“Light-sensitive coloring materials” means coloring materials whose color (for example: a coloring material) or shade (for example: a mixture of coloring materials) in the formulation is not stable after exposure of the composition to light. The stability of the color or shade can be observed with the naked eye by an expert colorist or measured with a spectrocolorimeter by calculating the color difference ΔE₀₀ between 2 samples (irradiated vs. control). A color or shade that is not light stable is a color or shade that loses intensity and/or saturation. The surface of the composition is also said to be discolored in the light. To select coloring materials that are light sensitive and capable of being used in the compositions of the invention, it is possible in particular to use the following test (otherwise called ‘Sun Test’) which aims at identifying coloring materials whose color or shade in formulation is impacted by exposure to light, for example irradiation for 16 hours:
The coloring material is generally pre-dispersed (for pigments) or solubilized (for colorants) in an oil then the mixture (otherwise called ‘pigment paste’) is dispersed homogeneously in an oily or fatty base comprising for example hydrocarbon oils and waxes as described below by way of illustration;
The composition obtained in the previous step is then packaged in two bowls, one serving as a control and the other being subjected to irradiation, for example for 16 hours; irradiation is carried out using a Sun Test XLS type apparatus, at a power of 500 W/m², for 16 hours, with a Store light reduced IR filter;
The shades of the irradiated composition versus the control composition (non-irradiated) are evaluated by an expert colorist who observes and describes the color or shade of each composition or by measuring a color difference ΔE₀₀ according to the protocol described below;
A shade that varies (for example: loses intensity and/or saturation, is discolored) in the irradiated condition compared to the control condition (for example a measurement of ΔE₀₀ greater than 1, in particular greater than 3, more particularly greater than 8, or even greater than 10 between the irradiated composition versus the non-irradiated control) indicates that the coloring material in the formulation is a light-sensitive coloring material, capable of being used in the present invention in association with the zinc oxide particles that will prevent this discoloration.

The parameter ΔE₀₀ is evaluated according to the following protocol.

The colorimetric parameters of the compositions are measured using a spectrocolorimeter such as a d/8° spectrocolorimeter (COLORi7800 X Rite).

The apparatus is used in specular mode included, with an aperture window of 17 mm, UV is excluded from the light source.

Spectral measurements are carried out on each sample before and after irradiation.

The colorimetric values are calculated in the CIELAB76 space, in which L* represents the color intensity, a* indicates the green/red color axis and b* the blue/yellow color axis, illuminating D65, observer 10°.

Color differences are calculated to evaluate the colorimetric evolution of the samples between the shade of the irradiated composition versus the control composition: difference in clarity DL* ab, chroma DC*ab, shade DH*ab (ISO/CIE 11664-4: 2019) and overall color difference ΔE₀₀ determined as defined by the CIE (ISO/CIE 11664-6:2014).

As an example for the implementation of this test, the following oily and fatty base can be used:

TABLE 1

| Ingredients (INCl name) | % by weight |
|---|---|
| POLYISOBUTENE | Qsp100 |
| OCTYLDODECANOL | 14.5 |
| DISTEARDIMONIUM HECTORITE | 1.7 |
| WAXES | 25 |
| CHOLESTEROL ESTERS | 8.3 |
| POLYGLYCEROL ESTER | 6 |
| CALCIUM CARBONATE | 5 |

This mixture is used as a dispersion base (once melted) for bichromatic pigment preparations with equivalent contents of TiO2 (opacifier) and coloring material to be tested. The mixture “Base+TiO2 pigment paste+Coloring material pigment paste” is adjusted in order to approach a level of shade saturation reflecting that existing in the products currently on the market; the texture is adjusted by adding a polyglycerol ester. Pigment pastes are dispersions of coloring materials in a polyglycerol ester, of known concentration, prepared extemporaneously. For example, 60% of this base is mixed with 5% of a TiO2 pigment paste (3% of TiO2 pre-dispersed in the polyglycerol ester), 10% of pigment paste Red 28 Lake to be tested (3% of Red 28 Lake pre-dispersed in polyglycerol ester) and 25% polyglycerol ester (to bring the composition to 100%). This mixture is heated to 95° C. and homogenized with a rotor-stator to guarantee good color homogeneity. The product is poured at a temperature of around 90° C. into a mold. Said product is left for a few minutes at room temperature, then put in the freezer for 5 minutes. At the end, the product is either unmolded or left in its mold and left to rest for at least 24 hours before undergoing the Sun Test.

The steps of the protocol are followed and using the SunTest it is evaluated whether the irradiated shade varies compared to the control shade. Compositions for which a variation in color is observed indicate that the coloring material tested is light sensitive.

The Applicant in particular tested the colorants and organic pigments Red 28 Lake, Red 6 Salt, Blue 1 Lake and a mica-based nacres coated with Red 6 and showed a variation in shade according to this protocol, with the greatest variation for Red 28 Lake, the most light-sensitive.

Thus, among the “light-sensitive colorants and organic pigments (for example: lacquers)” which can be used according to the invention, mention can in particular be made of the compounds Blue 1, Blue 1 lake (CI 42090), Yellow 5, Yellow 5 lake (CI 19140), Yellow 6, Yellow 6 lake (CI 15985), Yellow 10, Yellow 10 lake (CI 47005), Yellow 11, Yellow 11 lake, Red 4, Red 4 lake (CI 14700), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), Red 30, Red 30 lake (CI 73360), Red 33, Red 33 lake (CI 15880), Red 34, Red 34 lake (CI 15880), Red 40, Red 40 Lake (CI 16035)), Orange 4, carmine, and/or their salts, in particular sodium or barium, such as Red 6 Na Salt Lake, Red 6 Ba Salt Lake, and mixtures thereof; in particular Blue 1, Blue 1 lake (CI 42090), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), and/or their salts, and mixtures thereof; preferably Red 27, Red 27 lake (CI 45410), Red 28, Red 28 lake (CI 45410), and/or their salts, and mixtures thereof.

“Lacquers” are organic pigments comprising at least one organic colorant precipitated on an inert support (otherwise called “base”). In particular, lacquers comprising a base chosen from alumina (Al), barium sulfate (Ba), or calcium rosinate (Ca) bases will be used. For example Red 28 Al lake, is a lacquer comprising an alumina base on which the Red 28 colorant is precipitated.

According to a more particular embodiment, the composition comprises at least one colorant or organic pigment (for example lacquer) chosen from the group consisting of Blue 1, Blue 1 lake (CI 42090), Red 6, Red 6 lake (15850), Red 7, Red 7 lake (CI 15850), Red 27, Red 27 lake (CI 45410), Red 28, Red 28 lake (CI 45410), and/or their sodium or barium salts, and mixtures thereof; preferably Red 27, Red 27 lake (CI 45410), Red 28, Red 28 lake (CI 45410), and/or their sodium or barium salts, and mixtures thereof; more preferably Red 28, Red 28 lake (CI 45410), and/or their sodium or barium salts, and mixtures thereof.

“Composite pigment” within the meaning of the invention means a pigment comprising a substrate and mineral and/or organic pigment(s) which are embedded and/or encapsulated in said substrate. The light-sensitive composite pigments, which can be used according to the invention, therefore comprise a substrate and at least one of the aforementioned light-sensitive colorants or organic pigments.

Generally speaking, composite pigments resulting from the encapsulation and/or incrustation of mineral and/or organic pigments in or on substrates or matrices of different natures provide new optical properties to the pigments: in particular a lot of transparency while providing blurring properties.

The substrate may be a matrix or a core comprising at least one inorganic material and/or at least one organic material.

Thus, the light-sensitive composite pigments according to the invention comprise an inorganic and/or organic substrate, in particular a silica, a borosilicate or an alumina, in which at least one of the aforementioned colorants or organic pigments are embedded or encapsulated, and mixtures thereof.

The inorganic material of the substrate of said composite pigment can be chosen from the group consisting of: mica, synthetic mica, talc, sericite, boron nitride, glass or borosilicate flakes, calcium carbonate, barium sulfate, titanium oxide, hydroxyapatite, silica, silicate, zinc oxide, magnesium sulfate, magnesium carbonate, magnesium trisilicate, aluminum oxide, aluminum silicate, alumina, calcium silicate, calcium phosphate, magnesium oxide, bismuth oxychloride, kaolin, mineral clay, synthetic clay, and mixtures thereof.

The organic material of the composite pigment substrate may be chosen from the group consisting of: polymethylmethacrylates (PMMA), polyamides, silicones, polyurethanes, polyethylenes, polypropylenes, polystyrenes, polysaccharides, cellulose, polyvinyl alcohols, polyvinyl resins, waxes, and mixtures thereof.

In a preferred embodiment of the invention, at least one of the colorants or organic pigments of said composite pigment is chosen from the light-sensitive colorants and organic pigments mentioned above and mixtures thereof and the substrate is chosen from silica, and more particularly spheres of silica, borosilicate, alumina, or mixtures thereof.

According to a particular embodiment, said composite pigment is treated on the surface in particular by a lipophilic treatment agent and the coating obtained can be continuous or discontinuous.

“Pearlescent pigments”, also called “nacres” or “reflective particles”, means pigments with multilayer structures (different from the aforementioned composite pigments which do not have a multilayer structure) comprising a substrate (for example: mica, bismuth oxychloride) covered with at least one of the colorants or organic pigments as previously mentioned, and whose size, structure and physical and chemical nature of the layers as well as their surface state, allows to reflect incident light and create highlighting points on the skin after application of said composition containing same.

As nacres which can be used according to the invention, mention may be made in particular of colored pearlescent pigments based on mica and at least one of the aforementioned colorants or organic pigments. In particular, mention can be made of nacres based on synthetic mica with Blue 1, Blue 1 lake (CI42090), Yellow 5, Yellow 5 lake (CI 19140), Red 6, Red 6 lake (15850), Red 7, Red 7 lake (CI 15850), Red 27, Red 27 lake (CI 45410), Red 28, Red 28 lake (CI 45410), or carmine, or mixtures thereof. Mention can be made of the nacres ranges with commercial reference Intenza®, Colorona®, Sunshine®, Syncrystal®, and Reflecks®.

Thus, according to a particular embodiment, the composition of the invention comprises one or more light-sensitive coloring materials, chosen from the group consisting of:
(i) Colorants and organic pigments chosen from the group consisting of Blue 1, Blue 1 lake (CI 42090), Yellow 5, Yellow 5 lake (CI 19140), Yellow 6, Yellow 6 lake (CI 15985), Yellow 10, Yellow 10 lake (CI 47005), Yellow 11, Yellow 11 lake, Red 4, Red 4 lake (CI 14700), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), Red 30, Red 30 lake (CI 73360), Red 33, Red 33 lake (CI 15880), Red 34, Red 34 lake (CI 15880), Red 40, Red 40 Lake (CI 16035), Orange 4, carmine, and/or their salts, in particular Sodium or Barium, such as Red 6 Na Salt Lake, Red 6 Ba Salt Lake, and mixtures thereof; in particular Blue 1, Blue 1 lake (CI 42090), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), and/or their salts, and mixtures thereof; preferably Red 27, Red 27 lake (CI 45410), Red 28, Red 28 lake (CI 45410), and/or their salts, and mixtures thereof;
(ii) Composite pigments comprising an inorganic and/or organic substrate, in particular a silica, a borosilicate or an alumina, in which at least one of the aforementioned colorants or organic pigments are embedded or encapsulated, and mixtures thereof;
(iii) Pearlescent pigments comprising a substrate, in particular mica, covered with at least one of said colorants or organic pigments mentioned above, and mixtures thereof; in particular pearlescent pigments comprising a synthetic mica covered with a colorant or organic pigment chosen from Blue 1, Blue 1 lake (CI 42090), Yellow 5, Yellow 5 lake (CI 19140), Red 6, Red 6 lake (15850), Red 7, Red 7 lake (CI 15850), Red 27, Red 27 lake (CI 45410), Red 28, Red 28 lake (CI 45410), carmine, and mixtures thereof;
(iv) and mixtures thereof;
more preferably the colorants or organic pigments chosen from Red 27, Red 27 lake (CI 45410), Red 28, and Red 28 lake (CI 45410), or a composite pigment comprising Red 27, Red 27 lake (CI 45410), Red 28, or Red 28 lake (CI 45410), or a nacre comprising Red 27, Red 27 lake (CI 45410), Red 28, or Red 28 lake (CI 45410), and mixtures thereof.

In particular, the total content of light-sensitive coloring material(s) in the compositions of the invention will range from 0.001% to 50% by weight, in particular from 0.01% to 40% by weight, in particular from 0.1% to 20% relative to the total weight of said composition.

For a compacted powder composition (for example eye shadow, foundation powder), the total content of light-sensitive coloring material(s) will generally range from 0.001% to 50%, preferably from 0.01% to 40% by weight relative to the total weight of the composition.

For an anhydrous solid composition for the skin and different from a powder (for example complexion corrector), the total content of light-sensitive coloring material(s) will generally range from 0.001% to 30%, preferably 0.01% to 20% by weight relative to the total weight of the composition.

For an anhydrous solid composition for the lips (for example lip stick), the total content of light-sensitive coloring material(s) will generally range from 0.001% to 30%, preferably from 0.01% to 20% by weight relative to the total weight of the composition.

According to a particular embodiment, the composition of the invention further comprises another additional ingredient chosen from additional coloring materials, fillers, lipophilic gelling agents, pasty fatty substances, and mixtures thereof.

### Additional Coloring Materials

The composition may also comprise one or more additional coloring materials, different from the light-sensitive powdery coloring materials as defined above.

These additional coloring materials can be chosen from organic or inorganic coloring materials, soluble or insoluble in the fatty phase of the composition, composite pigments, nacres, and mixtures thereof.

According to a particular embodiment, the additional colorant(s) are chosen in particular from mineral pigments different from zinc oxides; organic pigments; composite pigments; nacres; colorants, and mixtures thereof.

Among the mineral pigments different from zinc oxides, mention can be made of iron oxides, in particular black, yellow, red and brown iron oxides; manganese violet; ultramarine blue, chromium oxide, and ferric blue.

Preferably, the composition will comprise one or more iron oxides.

Among the “additional colorants or organic pigments”, mention can be made of Green 3, Green 5, Orange 10, Violet 2, Red 17, Green 6, Sudan red, Sudan brown, Orange 5, and quinoline yellow, and/or their lacquers, and/or their salts, and mixtures thereof.

Among the “additional composite pigments”, mention can be made of those formed from a substrate and inorganic pigments such as iron oxides or organic ones not sensitive to light such as those mentioned above.

Among the “additional nacres”, mention can in particular be made of colored pearlescent pigments, such as mica with iron oxides and with or without TiO2, mica with an additional organic pigment of the aforementioned type, as well as pigments based on bismuth oxychloride. Mention can be made of the nacres ranges with commercial reference Reflecks®, Ronastar®, Timiron® and Syncrystal®. Pearlescent pigments may also have a base of silica, alumina, alumina hydroxide, synthetic mica, tin oxide, borosilicate (for example: calcium sodium borosilicate, calcium aluminum borosilicate or cellulose or cellulose derivatives).

According to a particular embodiment, the composition of the invention comprises one or more additional coloring materials chosen from the group consisting of iron oxides, additional organic pigments, additional nacres, and mixtures thereof.

In particular, the total content of coloring materials (including light-sensitive and non-light sensitive coloring materials) in the composition will range from 0.001% to 60% by weight, preferably from 0.01% to 50% by weight, even more preferably from 0.1% to 40% by weight relative to the total weight of the composition.

For a compacted powder composition (for example eye shadow, foundation powder), the total content of coloring material(s) will generally range from 0.001% to 50%, preferably from 0.01% to 40% by weight relative to the total weight of the composition.

For an anhydrous solid composition for the skin and different from a powder (for example complexion corrector), the total content of coloring material(s) will generally range from 0.001% to 30%, preferably from 0.01% to 20% by weight relative to the total weight of the composition.

For an anhydrous solid composition for the lips (for example: lip stick), the total content of coloring material(s) will generally range from 0.001% to 30%, preferably from 0.01% to 20% by weight relative to the total weight of the composition.

The composition of the invention may further comprise one or more additional fillers (different from the zinc oxide particles).

### Fillers

The fillers are used in particular to modify the rheology or texture of the composition and/or to confer a mattifying and/or blurring effect.

For the purposes of the invention, “fillers” means particles of any shape, colorless or white, of mineral or organic nature, natural or synthetic, which are in a form (platelet, spherical or oblong), insoluble and dispersed in the middle of the composition. The additional fillers, different from the zinc oxide particles, are chosen in particular from micas, of natural or synthetic origin; talc; bismuth oxychloride; silica; boron nitride; kaolin; calcium carbonate, magnesium carbonate and hydrocarbonate; zinc, magnesium or lithium stearate, zinc laurate, magnesium myristate; powders of synthetic polymers, such as polyethylene, polyesters, polyamides (for example nylon); polyacrylic or polymethacrylic acid powders, silicone powders; cellulose powders; glass and ceramic beads; powders of organic materials of natural origin such as corn, wheat, rice or cotton starches, crosslinked or not; hydroxyapatite and mixtures thereof.

According to a particular embodiment, the composition of the invention does not comprise talc.

The filters may or may not be treated on the surface, in particular by a lipophilic or hydrophobic agent; such as those described previously for zinc oxide particles.

According to a particular embodiment, the filler is not treated on the surface.

According to another particular embodiment, the filler is treated on the surface with a lipophilic agent such as those described above.

According to a particular embodiment, the composition of the invention comprises at least boron nitride particles as filler. The presence of boron nitride particles is advantageous in a solid anhydrous composition comprising zinc oxide particles, to obtain optimal hardness.

### Boron Nitride Particles

The particles of boron nitride particles which can be used according to the invention are generally of lamellar or platelet shape. ‘Lamellar particle’ or ‘platelet particle’ means that the thickness of the particle is less than the width of the particle, in particular the ratio between the width and the thickness of the particle ranges from 2 to 100.

Boron nitride particles generally have the INCI name: Boron nitride.

The boron nitride particles which can be used according to the invention generally have an average size ranging from 0.5 to 20 μm, in particular from 1 to 10 μm, measured according to a laser diffraction distribution method described previously.

Mention can in particular be made of the following commercial references: BORON NITRIDE CC and SOFTOUCH CC from the company SAFIC ALCAN/Momentive, BORON NITRIDE SHP from the company IWASE/Mizushima, RONAFLAIR from Merck, CARESS BN from Kobo.

The particles of boron nitride particles may or may not be treated with a lipophilic treatment agent.

According to a particular embodiment, the boron nitride particles are not treated.

According to a particular embodiment, the boron nitride particles are not in the form of composite particles.

According to a particular embodiment, the content of boron nitride particles in the composition of the invention will range in particular from 0.1% to 50%, in particular from 2% to 30% by weight relative to the total weight of the composition.

According to a particular embodiment, the composition further comprises at least one additional filler, different from zinc oxide particles and boron nitride particles. Examples of additional fillers are listed above.

The total content of filler(s) in the composition of the invention will range in particular from 0.01% to 50%, in particular from 0.1 to 45% by weight relative to the total weight of the composition.

For a compacted powder composition (for example: eye shadow, foundation powder), the total content of filler(s) will generally range from 1 to 50% by weight, in particular from 5 to 45% by weight relative to the total weight of the composition.

For an anhydrous solid composition for the skin and different from a powder (for example complexion corrector), the total filler content will generally range from 0.5 to 30% by weight, in particular from 1 to 25% by weight relative to the total weight of the composition.

For an anhydrous solid composition for the lips (for example: lip stick), the total content of filler(s) will generally range from 0.01 to 30% by weight, in particular from 1 to 25% by weight relative to the total weight of the composition.

### Oily or Fatty Phase

The composition may comprise one or more oils and optionally also an ingredient chosen from lipophilic gelling agents, waxes, pasty fatty substances and mixtures thereof.

“Oil” means, within the meaning of the invention, a fatty substance, not soluble in water, liquid at 25° C. and atmospheric pressure.

As oils which can be used in the invention, mention may in particular be made of hydrocarbon oils, silicone oils and mixtures thereof. According to a particular embodiment, the composition of the invention comprises one or more hydrocarbon oils.

As hydrocarbon oils, mention may in particular be made of hydrocarbon oils of vegetable or mineral origin, linear or branched C8-C19 alkanes, synthetic C₁₀-C₄₀ ethers, synthetic C₁₀-C₄₀ esters, C₁₂-C₂₆ fatty alcohols optionally branched and/or unsaturated, higher C₁₂-C₂₂ fatty acids optionally branched and/or unsaturated, mono- and/or polyesters of fatty acids and/or fatty alcohols and mixtures thereof.

As an example of oils of vegetable origin, mention is made of soybean oil, grape seed oil, sesame oil, corn oil, rapeseed oil, sunflower oil, cottonseed oil, avocado oil, olive oil, castor seed oil, jojoba seed oil, peanut oil, sweet almond oil, palm oil, pumpkin oil, hazelnut oil, apricot kernel oil, wheat germ oil, calophyllum oil, sesame oil, macadamia oil, coconut oil, cereal germ oil; candlenut oil, thistle oil, candellila oil, safflower oil, shea butter, and mixtures thereof.

As an example of a C10-C26 aliphatic fatty alcohol, mention is made of linear or branched, saturated, C10-C26, more preferably C16-C20 such as 2-octyldodecanol, aliphatic monoalcohols.

As an example of linear or branched hydrocarbons, mention is made of paraffin oils and their derivatives, petroleum jelly, mineral oils (mixtures of hydrocarbon oils derived from petroleum), polybutene, hydrogenated polyisobutene, polyisoprene, polydecenes such as hydrogenated polydecene, or else among linear, branched and/or cyclic alkanes.

As an example of possibly branched and/or unsaturated fatty acids, mention is made of myristic acid, palmitic acid, stearic acid, behenic acid, oleic acid, linoleic acid, linolenic acid or isostearic acid, and mixtures thereof.

As an example of esters, mention is made of monoesters or polyesters of fatty acids whose linear or branched fatty chain contains from 6 to 30 carbon atoms such as those mentioned above, or mixtures thereof, and fatty alcohols whose fatty chain contains from 3 to 30 carbon atoms, and in particular mono- and polyesters of hydroxy acids and fatty alcohols, esters of benzoic acid and fatty alcohols, polyesters of polyols, dipentaerythrityl C5-C9 esters, or polyglycerol polyesters, trimethylolpropane polyesters, propylene glycol polyesters, hydrogenated castor oil polyesters, and more particularly isononyl isononanoate, stearyl octanoate, isopropyl palmitate, isopropyl myristate, butyl stearate, hexyl laurate, 2-ethylhexyl palmitate, 2-hexyldecyl laurate, 2octyldecyl palmitate, 2-octyldodecyl myristate, 2-diethylhexyl succinate, diisostearyl malate, 2-octyl-dodecyl lactate, glycerin triisostearate or diglycerin triisostearate; diisostearyl malate, octyldodecyl stearyl stearate, cetearyl isononanoate, diisopropyl adipate, caprylic capric triglyceride, glycerol tricaprocaprylate, isocetyl stearoyl stearate, alkyl benzoate, (di)pentaerythrityl, pentaerythrityl tetraisostearate, dipentaerythrityl pentaisononanoate, bis-diglyceryl polyacyladipate-2, trimethylolpropane triethylhexanoate, triisostearate trimethylolpropane, propylene glycol dibenzoate, propylene glycol dioctanoate, mixtures of triglycerides obtained by esterification of glycerol with capric acid and caprylic acid, non-oxyalkylenated polyglycerol esters such as polyglyceryl-2 triisostearate, polyglyceryl-2 isostearate or polyglyceryl-2 diisostearate, isostearyl lactate, octyldodecyl hydroxystearate, diisostearyl malate, glycerin stearate; diethyleneglycol diisononanoate; jojoba esters, i polyglyceryl polyricinoleates such as polyglyceryl-3 polyricinoleates, polyglyceryl-5 polyricinoleates, polyglyceryl-6 polyricinoleates, and polyglyceryl-10 polyricinoleates (in their INCI names), and mixtures thereof.

The oil content can range from 0.2 to 90%, preferably from 0.5% to 80%, better still from 1% to 70% by weight relative to the total weight of the composition.

For a compacted powder composition (for example: eye shadow, foundation powder), the total content of oil(s) will generally range from 0.2 to 50% by weight, in particular from 2% to 15% by weight relative to the total weight of the composition.

For an anhydrous solid composition for the skin and different from a powder (for example complexion corrector), the total content of oil(s) will generally range from 10 to 90% by weight, in particular from 20 to 60% by weight relative to the total weight of the composition.

For an anhydrous solid composition for the lips (for example lip stick), the total content of oil(s) will generally range from 10 to 90% by weight, in particular from 20 to 60% by weight relative to the total weight of the composition.

According to a particular embodiment, the composition further comprises at least one lipophilic gelling agent.

### Lipophilic Gelling Agents

“Lipophilic gelling agent” according to the invention means a compound capable of gelling the fatty phase of a composition.

The lipophilic gelling agent can advantageously be chosen from natural or synthetic clays; modified natural micas such as aluminum, magnesium and potassium fluorosilicate; dextrin and fatty acid esters such as dextrin palmitate or dextrin myristate; tri-esters of C8 C30 fatty acid and of mono- or poly-glyceryl such as glyceryl tri(hydroxystearate) (INCI name: Trihydroxystearin).

The lipophilic gelling agent is preferably a natural or synthetic clay chosen from bentonites, in particular hectorites and montmorillonites, beidellites, saponites, nontronites, sepiolites, biotites, attapulgites, vermiculites and zeolites. Preferably, it can be chosen from hectorites. Even more preferably a hectorite modified with a quaternary alkylammonium chloride is used, said ammonium being substituted by at least one, and preferably at least two C14-C20 alkyl radicals, such as disteardimonium hectorite in which the ammonium comprises two methyls and two stearyls.

As a preferred fat-soluble gelling agent, mention may be made in particular of a modified hectorite dispersed in an oil, for example the reference BENTONE GEL from Elementis with the INCI name Octyldodecanol (and) Disteardimonium Hectorite (and) Propylene Carbonate.

Thus, according to a particular embodiment, the composition of the invention further comprises a lipophilic gelling agent, in particular a mineral gelling agent such as bentones, preferably a quaternary hectorite.

When the composition comprises one or more gelling agent(s), the total content of lipophilic gelling agent(s) may range from 0.5 to 5%, in particular from 2 to 4% by weight relative to the total weight of the composition.

According to a particular and preferred embodiment, the composition of the invention comprises one or more waxes.

### Waxes

“Wax”, within the meaning of the present invention, means a solid compound at 25° C. which has a reversible solid/liquid state change and a melting temperature greater than 30° C., preferably greater than 45° C.

Mention can be made of microcrystalline waxes, paraffin waxes, polyethylene waxes, ozokerite, products comprising a mixture of polyethylene and alcohols containing 20 to 50 carbon atoms, silicone waxes in particular alkyl dimethicones, C20-C40 alkyl stearates, beeswax, waxes of plant origin, copolymers of maleic anhydride and alpha-olefin, waxes obtained by metallocene catalysis, and mixtures thereof.

According to a particular and preferred embodiment, the total content of wax(es) in the composition of the invention ranges from 0.1% to 30% by weight, preferably from 0.5% to 25%, more preferably from 1% to 25%, preferably from 4 to 20% by weight, in particular from 5 to 15% by weight relative to the total weight of the composition.

For a compacted powder composition (for example: eye shadow, foundation powder), the total wax content will generally range from 0.1 to 10% by weight, preferably 0.2 to 5% by weight relative to the total weight of the composition.

For an anhydrous solid composition for the skin and different from a powder (for example: complexion corrector), the total wax content will generally range from 0.5% to 30% by weight, better still from 5% to 15% by weight relative to the total weight of the composition.

For an anhydrous solid composition for the lips (for example: lip stick), the total wax content will generally range from 0.5% to 30% by weight, better still from 5% to 15% by weight relative to the total weight of the composition.

According to a particular embodiment, the composition of the invention further comprises one or more pasty fatty substances.

### Pasty Fatty Substances

“Pasty fatty substance” means a non-crystalline fatty compound comprising, at a temperature of 25° C., a liquid fraction and a solid fraction.

According to the invention, the pasty fatty substance can be chosen from the group consisting of lanolin and its derivatives, polymeric silicone compounds, alkyl (meth)acrylate copolymers preferably having a C8-C30 alkyl group, homo and copolymer oligomers of vinyl esters having C8-C30 alkyl groups, homo and copolymer oligomers of vinyl ethers having C8-C30 alkyl groups, fat-soluble polyethers resulting from polyetherification between one or more C2-050 diols, ethylene oxide and/or propylene oxide copolymers with long chain C6-C30 alkylene oxides, diglycerol esters, arachidyl propionate, phytosterol esters, non-crosslinked polyesters resulting from the polycondensation between a diacid or a linear or branched C4-C50 carboxylic polyacid and a diol or a polyol, the ester resulting from the esterification reaction of hydrogenated castor oil with isostearic acid such as a mono di- or tri-isostearate of hydrogenated castor oil, a mixture of soy sterols and oxyethylenated (5 OE) oxypropylenated (5 OP) pentaerythritol, triglycerides of fatty acids and their derivatives, shea butter, cocoa butter, mango oil or butter, and mixtures thereof.

Mention may in particular be made of triglycerides of fatty acids and their derivatives, esters of polyol(s) and of fatty acid diacid dimer, or of one of its esters, esters of hydrogenated castor oil, oligomeric esters of glycerol, in particular diglycerol esters, vegetable butters and mixtures thereof.

The content of pasty fatty substances can range from 0.05 to 30% by weight relative to the total weight of the composition.

For a compacted powder composition (for example: eye shadow, foundation powder), the total content of pasty fatty substances will generally range from 0 to 10%, in particular from 0.2 to 5% by weight relative to the total weight of the composition.

For an anhydrous solid composition for the skin and different from a powder (for example: complexion corrector), the total content of pasty fatty substances will generally range from 0 to 30% by weight, in particular from 5 to 15% by weight relative to the total weight of the composition. For an anhydrous solid composition for the lips (for example: lip stick), the total content of pasty fatty substances will generally range from 0 to 30% by weight, in particular from 5 to 15% by weight relative to the total weight of the composition.

According to a particular embodiment, the composition of the invention is a solid composition comprising:
Zinc oxide particles,
One or more light-sensitive coloring materials, chosen from the group consisting of colorants and organic pigments (for example: lacquers), composite pigments comprising at least one of said colorants or organic pigments, pearlescent pigments comprising at least one of said colorants or organic pigments, and mixtures thereof,
Boron nitride particles,
One or more waxes, preferably in a total content ranging from 1 to 25% by weight, in particular from 4 to 20% by weight relative to the total weight of the composition,
Hydrocarbon oils,
Optionally a lipophilic gelling agent,
Optionally one or more additional coloring materials not sensitive to light, chosen from the group consisting of iron oxides, organic pigments, nacres, and mixtures thereof,
and characterized in that it comprises less than 3% by weight of titanium dioxide relative to the total weight of the composition, in particular less than 1% by weight of titanium dioxide, or is even free of titanium dioxide.

According to a particular and preferred embodiment, the composition of the invention is a solid composition comprising:
Zinc oxide particles, in particular in a content of 0.1 to 50% by weight, in particular 1 to 30% by weight or even 2 to 20% by weight, relative to the total weight of the composition,
One or more light-sensitive coloring materials, chosen from the group consisting of colorants or organic pigments (for example: lacquers), composite pigments comprising at least one of said colorants or organic pigments, pearlescent pigments comprising at least one of said colorants or organic pigments, and mixtures thereof, in particular in a content of 0.001% to 30%, better still from 0.01% to 20% by weight relative to the total weight of the composition,
Boron nitride particles, in particular in a content of 0.1 to 50% by weight, in particular from 2 to 30% by weight relative to the total weight of the composition,
One or more waxes in a total content ranging from 1 to 25% by weight, in particular from 4 to 20% by weight relative to the total weight of the composition,
Hydrocarbon oils,
One or more lipophilic gelling agents,
One or more additional coloring materials not sensitive to light, chosen from the group consisting of iron oxides, organic pigments, nacres, and mixtures thereof,
and characterized in that it is free of titanium dioxide.

According to another particular embodiment, the composition of the invention is a fluid or liquid composition comprising:
Zinc oxide particles,
One or more light-sensitive coloring materials, chosen from the group consisting of colorants and organic pigments (for example: lacquers), composite pigments comprising at least one of said colorants or organic pigments, pearlescent pigments comprising at least one of said colorants or organic pigments, and mixtures thereof,
Hydrocarbon oils,
Optionally a lipophilic gelling agent,
Optionally one or more additional coloring materials not sensitive to light, chosen from the group consisting of iron oxides, organic pigments, nacres, and mixtures thereof,
and characterized in that it comprises less than 3% by weight of titanium dioxide relative to the total weight of the composition, in particular less than 1% by weight of titanium dioxide, or is even free of titanium dioxide.

According to a particular and preferred embodiment, the composition of the invention is a fluid or liquid composition comprising:
Zinc oxide particles, in particular in a content of 0.1 to 50% by weight, in particular 1 to 30% by weight, or even 2 to 20% by weight, relative to the total weight of the composition,
One or more light-sensitive coloring materials, chosen from the group consisting of colorants and organic pigments (for example: lacquers), composite pigments comprising at least one of said colorants or organic pigments, pearlescent pigments comprising at least one of said colorants or organic pigments, and mixtures thereof, in particular in a content of 001% to 50%, in particular from 0.01 to 40%, or even from 0.01% to 20% by weight relative to the total weight of the composition,
Hydrocarbon oils,
One or more lipophilic gelling agents,
One or more additional coloring materials not sensitive to light, chosen from the group consisting of iron oxides, organic pigments, nacres, and mixtures thereof, and characterized in that it is free of titanium dioxide.

According to another particular embodiment, the composition of the invention is a powder, in particular a loose powder, comprising:
Zinc oxide particles,
One or more light-sensitive coloring materials, chosen from the group consisting of colorants and organic pigments (for example: lacquers), composite pigments comprising at least one of said colorants or organic pigments, pearlescent pigments comprising at least one of said colorants or organic pigments, and mixtures thereof,
Hydrocarbon oils,
Optionally a lipophilic gelling agent,
Optionally one or more additional coloring materials not sensitive to light, chosen from the group consisting of iron oxides, organic pigments, nacres, and mixtures thereof,
and characterized in that it comprises less than 3% by weight of titanium dioxide relative to the total weight of the composition, in particular less than 1% by weight of titanium dioxide, or is even free of titanium dioxide.

According to a particular and preferred embodiment, the composition of the invention is a powder, in particular a loose powder, comprising:
Zinc oxide particles, in particular in a content of 0.1 to 50% by weight, in particular 1 to 30% by weight, or even 2 to 20% by weight relative to the total weight of the composition,
One or more light-sensitive coloring materials, chosen from the group consisting of colorants and organic pigments (for example: lacquers), composite pigments comprising at least one of said colorants or organic pigments, pearlescent pigments comprising at least one of said colorants or organic pigments, and mixtures thereof, in particular in a content of 0.001% to 50%, in particular from 0.01% to 40%, or even from 0.1 to 20% by weight relative to the total weight of the composition,
Hydrocarbon oils,
One or more lipophilic gelling agents,
One or more additional coloring materials not sensitive to light, chosen from the group consisting of iron oxides, organic pigments, nacres, and mixtures thereof,
and characterized in that it is free of titanium dioxide.

In particular in these different particular and preferred embodiments, the light-sensitive coloring materials are chosen from the group consisting of:
(i) Colorants and organic pigments chosen from the group consisting of Blue 1, Blue 1 lake (CI 42090), Yellow 5, Yellow 5 lake (CI 19140), Yellow 6, Yellow 6 lake (CI 15985), Yellow 10, Yellow 10 lake (CI 47005), Yellow 11, Yellow 11 lake, Red 4, Red 4 lake (CI 14700), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), Red 30, Red 30 lake (CI 73360), Red 33, Red 33 lake (CI 15880), Red 34, Red 34 lake (CI 15880), Red 40, Red 40 Lake (CI 16035), Orange 4, carmine, and/or their salts, in particular Sodium or Barium salts, such as Red 6 Na Salt Lake, Red 6 Ba Salt Lake, and mixtures thereof; in particular Blue 1, Blue 1 lake (CI 42090), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), and/or their salts, and mixtures thereof; preferably Red 27, Red 27 lake (CI 45410), Red 28, Red 28 lake (CI 45410), and/or their salts, and mixtures thereof;
(ii) Composite pigments comprising an inorganic and/or organic substrate, in particular a silica, a borosilicate or an alumina, in which at least one of the aforementioned colorants or organic pigments are embedded or encapsulated, and mixtures thereof;
(iii) Pearlescent pigments comprising a substrate, in particular mica, covered with at least one of said colorants or organic pigments mentioned above, and mixtures thereof; in particular pearlescent pigments comprising a synthetic mica covered with a colorant or organic pigment chosen from Blue 1, Blue 1 lake (CI 42090), Yellow 5, Yellow 5 lake (CI 19140), Red 6, Red 6 lake (15850), Red 7, Red 7 lake (CI 15850), Red 27, Red 27 lake (CI 45410), Red 28, Red 28 lake (CI 45410), carmine, and mixtures thereof;
(iv) and mixtures thereof;

more preferably the colorants or organic pigments chosen from Red 27, Red 27 lake (CI 45410), Red 28, and Red 28 lake (CI 45410), or a composite pigment comprising Red 27, Red 27 lake (CI 45410), Red 28, or Red 28 lake (CI 45410), or a nacre comprising Red 27, Red 27 lake (CI 45410), Red 28, or Red 28 lake (CI 45410), and mixtures thereof.

### Galenic

The composition of the invention is a care and/or make-up composition for keratinous materials, in particular a composition for the skin, eyelids, eyelashes, or lips, preferably eyelids, eyelashes and lips.

The composition may be in the form of a loose powder, a fluid foundation, a lip gloss or in the form of a compact powder or a stick or a composition packaged in a pot or cup, in particular a lip gloss, a lip stick, a liner pencil, an eyelash pencil, an eye shadow, or a complexion corrector or concealer, preferably a lip gloss or a lip stick.

According to a first embodiment, the composition of the invention is in a liquid or fluid form. It will in particular be a fluid foundation or a lip gloss.

According to a first embodiment, the composition of the invention is in the form of a loose powder.

According to a particular embodiment, the composition of the invention is in the form of a compact powder.

According to another particular embodiment, the composition of the invention is in the form of a stick or of a composition packaged in a pot or a cup.

In particular it will be a compact powder for the eyelids (shadow or eyeshadow) or for the complexion.

According to another embodiment, it will be a stick for the lips or a composition in a pot or cup for the lips.

According to another particular embodiment, it will be a pencil liner.

According to another particular embodiment, it will be a stick or a pencil or a composition in a pot or cup, applicable with the finger or with a brush, for the eyelashes (for example: eyelash pencil).

According to another embodiment, it will be a stick or a composition in a pot or cup, applicable with the finger, with a sponge or with a brush, for the eyelids (for example: eyeshadow).

According to another embodiment, it will be a stick or a composition in a pot or cup, applicable with the finger, with a sponge or with a brush, for the skin of the face, in particular certain areas (for example: complexion corrector or concealer).

Thus, the composition of the invention is in particular in the form of a lip gloss or a lip stick, a liner pencil, an eyelash pencil, a blush or eyeshadow, or a complexion corrector or concealer, preferably a lip gloss or a lip stick.

### Cosmetic Method

The invention also relates to a cosmetic method for caring for and/or making up keratinous materials comprising the application, to said keratinous materials, in particular the skin, the eyelids, the eyelashes, or the lips, preferably the lips, of a composition according to the invention.

The compositions of the invention are as defined above.

According to a particular embodiment, the composition applied to the skin is in the form of a compact powder.

Preferably, the composition applied to the skin or the lips is in the form of a lip gloss, a lip stick, a liner pencil, an eyelash pencil, a blush or eyeshadow, or a complexion corrector or concealer, preferably a lip gloss or a lip stick.

The invention will now be illustrated in the following non-limiting examples. The % are expressed as % by weight of ingredient relative to the total weight of the composition, unless otherwise indicated.

### EXAMPLES

### Example 1: Effect of Zinc Oxide Particles on Color Protection

Several tests were carried out upstream to determine the coverage power of zinc oxide particles, compared to titanium dioxide, in order to be able to replace titanium dioxide in formulas, without impacting the desired coverage effect.

These results showed that, for an equivalent coverage power, in an anhydrous solid composition according to the invention, an amount of zinc oxide particles approximately 3 to 4 times greater than the amount of titanium dioxide was required for an equivalent coverage result.

Different colorants and organic pigments (for example: lacquers) and a nacre in a lipstick base were tested as described below:

TABLE 2

| Ingredients (INCl name) | % by weight |
|---|---|
| POLYISOBUTENE | Qsp100 |
| OCTYLDODECANOL | 14.5 |
| DISTEARDIMONIUM HECTORITE | 1.7 |
| WAXES | 25 |
| CHOLESTEROL ESTERS | 8.3 |
| POLYGLYCEROL ESTER | 6 |
| CALCIUM CARBONATE | 5 |

This mixture is used as a dispersion base (once melted) for bichromatic pigment preparations with equivalent contents of TiO2 (opacifier) and colorant to be tested. The mixture “Base+TiO2 pigment paste+Coloring material pigment paste” is adjusted in order to approach a level of shade saturation reflecting that existing in the products currently on the market; the texture is adjusted by adding a polyglycerol ester. Pigment pastes are dispersions of coloring materials in a polyglycerol ester, of known concentration, prepared extemporaneously.

To make the first line of the table easier to read, 60% of this base was mixed with 5% of a TiO2 pigment paste (3% of TiO2 pre-dispersed in the polyglycerol ester), 10% of pigment paste of Red 28 Lake to be tested (3% of Red 28 Lake pre-dispersed in the polyglycerol ester) and 25% of polyglycerol ester (to bring the composition to 100%). This mixture is heated to 95° C. and homogenized with a rotor-stator to guarantee good color homogeneity. The product is poured at a temperature of around 90° C. into a mold. Said product is left for a few minutes at room temperature, then put in the freezer for 5 minutes. At the end, the product is either unmolded or left in its mold and left to rest for at least 24 hours before undergoing the Sun Test.

### Color Evaluation by Sun Test:

The shade stability properties of the compositions to light were evaluated according to a ‘Sun Test’ protocol, in which the compositions are subjected to irradiation for 16 hours.

Each composition is poured into two pots of lipstick: a control and one which will be irradiated for 16 hours.

Irradiation is carried out using a Sun Test XLS type apparatus, at a power of 500 W/m², for 16 hours, with a Store light reduced IR filter.

The shades of each composition tested compared to the control composition (non-irradiated), are evaluated by a colorist who observes and describes the color variation of each composition.

The results are presented in the following table:

TABLE 3

| Tested | | | | | | |
|---|---|---|---|---|---|---|
| Pigments/ | | % expressed as pure material | | | | |
| nacres | | Pigment/ | | % TiO2 | % ZnO | |
| likely | | nacre likely | | (provided | (provided | |
| to be | | to be | | by the | by the | |
| sensitive | Formulas | sensitive | TiO2 in | pigment | pigment | SunTest |
| to light | Composition | to light | nacre | paste) | paste) | Visual evaluation after Sun Test |
| Red | Control | 3 | | 3 | | Strong discoloration |
| 28 Al | TiO2 | | | | | |
| lake | ZnO *4 | 3 | | | 12 | Reduction in discoloration (protective |
| | | | | | | effect of ZnO) |
| Red 6 | Control | 0.6 | | 3 | | Low discoloration |
| Salt | TiO2 | | | | | |
| | ZnO *4 | 0.6 | | | 12 | Reduction in discoloration (protective |
| | | | | | | effect of ZnO) |
| Blue 1 | Control | 3 | | 3 | | Medium discoloration |
| lake | TiO2 | | | | | |
| | ZnO *4 | 3 | | | 12 | Reduction in discoloration (protective |
| | | | | | | effect of ZnO) |
| Intenza | Control | 0.07 (In | 0.17 | 3 | | Medium discoloration |
| Orange | TiO2 | nacre) | | | | |
| mica- | ZnO *4 | 0.07 (In | 0.17 | | 12 | Reduction in discoloration (protective |
| Red 6 | | nacre) | | | | effect of ZnO) |

ZnO *4: the amount of ZnO is multiplied by 4 compared to the amount of TiO2 to obtain the same coverage power and to be ‘iso-tinted’ in the examples.

These results show that the zinc oxide particles (ZnO) particles have a protective effect on the discoloration of light-sensitive coloring materials.

### Example 2: Formulations

The following examples are illustrative and non-limiting examples of the invention, prepared according to methods well known to the cosmetic formulator.

### 2.1 Anhydrous Liquid Lip Gloss

TABLE 4

| INGREDIENTS | % by weight |
|---|---|
| POLYBUTENE | 20 |
| TRIDECYL TRIMELLITATE | 6 |
| PENTAERYTHRITYL TETRAISOSTEARATE | 10 |
| VEGETABLE FATTY ESTERS | 15 |
| COLORANTS | 0.1 |
| RED 27 LAKE | 0.01 |
| ZINC OXIDE PARTICLES | 0.2 |
| HYDROGENATED POLYISOBUTENE | QSP 100 |

### 2.2 Compact Powder

TABLE 5

| INGREDIENTS (INCl NAMES) | % by weight |
|---|---|
| SYNTHETIC FLUORPHLOGOPITE | Qsp 100 |
| NACRE (mica and Red 6) | 2 |
| MICA | 10 |
| CALCIUM SODIUM BOROSILICATE | 10 |
| SILICA | 7.9 |
| CONSERVATIVE | sb |
| ZINC OXIDE PARTICLES | 5.7 |
| ZINC STEARATE | 0.5 |
| Lipophilic treated IRON OXIDES | 3.3 |
| CAPRYLIC/CAPRIC/SUCCINIC TRIGLYCERIDE | 3 |
| (MIGLYOL 829 MB) | |
| BUTYLENE GLYCOL DICAPRYLATE/DICAPRATE | 5.5 |
| (DERMOFEEL BGC MB) | |
| POLYGLYCERYL-2 TRIISOSTEARATE | 2 |
| GLYCOLS | 2.5 |

The compact powder has good shade stability to light. Applied to the skin of the face, it has good coverage and color properties.

### 2.3 Anhydrous Lip Stick

TABLE 6

| INGREDIENTS | % by weight |
|---|---|
| SILICA | 5.0 |
| POLYETHYLENE WAX | 5.5 |
| CANDELILLA WAX | 3.0 |
| SHEA BUTTER | 1.5 |
| IRON OXIDE (red) | 5.0 |
| RED 28 LAKE | 2.0 |
| BORON NITRIDE PARTICLES | 0.2 |
| ZINC OXIDE PARTICLES | 3.0 |
| HYDROGENATED POLYDECENE | QSP100 |

The lipstick has good color stability to light. Applied to the lips, the lipstick provides good coverage and color properties.

### 2.4 Anhydrous Eyeshadow

TABLE 7

| Ingredients (INCl Name) | % by weight |
|---|---|
| SILICA | 15 |
| SYNTHETIC FLUORPHLOGOPITE | 10 |
| IRON OXIDE | 8 |
| ZINC OXIDE PARTICLES | 10 |
| NACRES (mica and Red 7 lake) | 2 |
| OILS AND WAXES | Qsp 100 |

The eye shadow, packaged in a cup, has good color stability in the light. Applied to the eyelids, the eyeshadow provides coverage and a pearly effect.

1.-11. (canceled)

12. A care and/or make-up cosmetic composition for keratinous materials, comprising, in a physiologically acceptable medium:
(i) Zinc oxide, and
(ii) One or more light-sensitive coloring materials, chosen from the group consisting of colorants and organic pigments, composite pigments comprising at least one of said colorants or organic pigments, pearlescent pigments comprising at least one of said colorants or organic pigments, and mixtures thereof,
characterized in that it comprises less than 3% by weight of titanium dioxide relative to the total weight of the composition.

13. The cosmetic composition according to claim 12, which is an anhydrous composition.

14. The cosmetic composition according to claim 12, wherein the total content of light-sensitive coloring material(s) ranges from 0.001 to 50% by weight relative to the total weight of the composition.

15. The cosmetic composition according to claim 12, wherein the light-sensitive coloring material is chosen from the group consisting of:
(i) Colorants and organic pigments chosen from the group consisting of Blue 1, Blue 1 lake (CI 42090), Yellow 5, Yellow 5 lake (CI 19140), Yellow 6, Yellow 6 lake (CI 15985), Yellow 10, Yellow 10 lake (CI 47005), Yellow 11, Yellow 11 lake, Red 4, Red 4 lake (CI 14700), Red 6, Red 6 lake (15850), Red 7, Red 7 lake (CI 15850), Red 21, Red 21 lake (CI 45380), Red 22, Red 22 lake (CI 45380), Red 27, Red 27 lake (CI 45410), Red 28, Red 28 lake (CI 45410), Red 30, Red 30 lake (CI 73360), Red 33, Red 33 lake (CI 15880), Red 34, Red 34 lake (CI 15880), Red 40, Red 40 Lake (CI 16035), Orange 4, carmine, and/or their salts;
(ii) Composite pigments comprising an inorganic and/or organic substrate, in which at least one of the aforementioned colorants or organic pigments are embedded or encapsulated, and mixtures thereof,
(iii) Pearlescent pigments comprising a substrate, covered with at least one of said colorants or organic pigments mentioned above, and mixtures thereof;
(iv) and mixtures thereof.

16. The cosmetic composition according to claim 15, wherein the light-sensitive colorant is chosen from the group made up of:
(i) Colorants and organic pigments chosen from Red 27, Red 27 lake (CI 45410), Red 28, Red 28 lake (CI 45410), and/or their salts, and mixtures thereof;
(ii) Composite pigments comprising an inorganic and/or organic substrate selected from silica, borosilicate and alumina,
(iii) Pearlescent pigments comprising synthetic mica covered with a colorant or organic pigment chosen from Blue 1, Blue 1 lake (CI 42090), Yellow 5, Yellow 5 lake (CI 19140), Red 6, Red 6 lake (15850), Red 7, Red 7 lake (CI 15850), Red 27, Red 27 lake (CI 45410), Red 28, Red 28 lake (CI 45410), carmine, and mixtures thereof,
(iv) and mixtures thereof.

17. The cosmetic composition according to claim 12, wherein the content of zinc oxide particles ranges from 0.1 to 50% by weight of zinc oxide, relative to the total weight of the composition.

18. The cosmetic composition according to claim 12, wherein it comprises less than 1% by weight of titanium dioxide relative to the total weight of the composition.

19. The cosmetic composition according to claim 18, which is free of titanium dioxide.

20. The cosmetic composition according to claim 12, which is a powder.

21. The cosmetic composition according to claim 12, which is a fluid or liquid composition.

22. The cosmetic composition according to claim 12, which it is a solid composition.

23. The cosmetic composition according to claim 12, wherein it further comprises another additional ingredient chosen from additional coloring materials, additional fillers, lipophilic gelling agents, waxes, pasty fatty substances, and mixtures thereof.

24. The cosmetic composition according to claim 12, in the form of a loose powder, a fluid foundation, a lip gloss or in the form of a compact powder or a stick or a composition packaged in a pot or cup.

25. The cosmetic composition according to claim 24, which is a lip gloss, a lip stick, a liner pencil, an eyelash pencil, an eye shadow, or a complexion corrector or concealer.

26. The cosmetic composition according to claim 25, which is a lip gloss or a lip stick.

27. A cosmetic method for caring for and/or making up keratinous materials, comprising the application to said keratinous materials of a composition as defined in claim 12.

28. The cosmetic method according to claim 27, wherein the composition is applied onto the skin, the eyelids, the eyelashes or the lips.
